(19) United States
(12) Patent Application Publication
(10) Pub. No.: US 2026/0222587 A1
(43) Pub. Date: Jul. 30, 2026

(54) VIDEO COMPRESSION AND TRANSMISSION DEVICE AND METHOD FOR REMOTE MEDICAL SYSTEM

(71) Applicant: KWANGWOON NIVERSITY INDUSTRY-ACADEMIC COLLABORATION FOUNDATON, Seoul (KR)

(72) Inventors: Dong Gyu SIM, Seoul (KR); See Nae PARK, Seoul (KR)

(21) Appl. No.: 19/143,351

(22) PCT Filed: Oct. 25, 2023
(86) PCT No.: PCT/KR2023/016683

§ 371 Date: Jun. 25, 2025

(30) Foreign Application Priority Data
Dec. 26, 2022 (KR) 10-2022-0184848

(51) Int. Cl. H04N 19/14; G06V 10/40; G06V 10/764; H04N 19/107; H04N 19/124; H04N 19/13; H04N 19/17; H04N 19/60

(52) U.S. Cl. CPC H04N 19/14; G06V 10/40; G06V 10/764; H04N 19/107; H04N 19/124; H04N 19/13; H04N 19/17; H04N 19/60; G06V 2201/03

(57) ABSTRACT

A video compression and transmission device and method for a remote medical system disclosed herein obtain feature information and structure information about an input image, determine a prediction method and a quantization parameter on the basis of the feature information and the structure information, and image-encode the input image on the basis of the compression method and the quantization parameter. The feature information includes at least one of the type of the input image, information about the behavior of an object in the image, or information about the composition of the image, and the structure information may be information obtained by classifying areas of the input image as a video conference image, an X-ray image, and a CT image on the basis of the feature information.

### TECHNICAL FIELD

The present invention relates to a technology for effectively encoding and decoding medical images and video images in a remote medical system, and more specifically, relates to a technology for effectively encoding and decoding images by using learned data.

### BACKGROUND ART

Recently, the need for remote medicine has increased due to social phenomena such as the pandemic. In response to this demand, the remote medicine service has been already implemented, and a system is being developed by using an international standard such as DICOM, etc. which is currently used in medical data systems inside hospitals. The technologies for medical images have been developed based on methods and devices for lossless compression and image quality improvement rather than applying an efficient compression technology by considering storage and transmission such as a general video or a still image due to the characteristics of their data. However, in a system where transmission can be performed to the outside of a hospital such as remote medicine, in addition to the existing processing technology for medical images, a technology that considers the transmission network will be required. In addition, other than basic medical data, the technology will also be required to process additional data for smooth communication between patients and medical personnel or between medical personnel.

### DISCLOSURE

### Technical Problem

Some embodiments of the present invention is to provide an efficient compression and processing method and device for compressing and transmitting an image and medical data for a video conference between users.

However, technical problems to be achieved by this embodiment are not limited to technical problems described above, and other technical problems may exist.

### Technical Solution

A video compression and transmission device, method and recording medium for the remote medical system of the present disclosure obtain feature information and structure information of an input image, determine a prediction method and a quantization parameter based on the feature information and the structure information, and image-encode the input image based on the compression method and the quantization parameter, wherein the feature information includes at least one of a type of an image for the input image, a behavior of an object in an image or configuration information of the image, and the structure information may be information obtained by classifying areas of the input image into a video conference image, an X-ray image and a CT image based on the feature information.

In a video compression and transmission device, method and recording medium for the remote medical system of the present disclosure, the image encoding may be performed based on a coding structure of a higher level than a unit in which the input image is encoded.

In a video compression and transmission device, method and recording medium for the remote medical system of the present disclosure, the coding structure of the higher level may be determined based on the feature information and the structure information.

In a video compression and transmission device, method and recording medium for the remote medical system of the present disclosure, the higher level may be any one of a picture, a sub-picture, a tile and a slice.

In a video compression and transmission device, method and recording medium for the remote medical system of the present disclosure, the prediction method may be any one of intra prediction, inter prediction, intra block copy and a mixed prediction method of intra prediction and inter prediction.

In a video compression and transmission device, method and recording medium for the remote medical system of the present disclosure, the prediction method may be determined by considering at least one of whether a resolution is changed, whether lossy compression is performed according to whether the resolution is changed or a compression ratio.

### Advantageous Effect

According to the problem solutions of the present invention described above, the efficiency of a decoder may be increased by effectively compressing an image in a remote medical image where medical information and a video conference image exist in combination.

### BRIEF DESCRIPTION OF DRAWINGS

FIG. 1 conceptually shows a remote medical system according to an embodiment of the present invention.

FIG. 2 shows an image processing process for remote medicine in a remote medical system according to an embodiment of the present invention.

FIG. 3 shows a processing process of an image classifier in a remote medical system according to an embodiment of the present invention.

FIG. 4 shows a processing process of an image quality determiner in a remote medical system according to an embodiment of the present invention.

FIG. 5 shows a processing process of an image compressor in a remote medical system according to an embodiment of the

### BEST MODE

A video compression and transmission device, method and recording medium for the remote medical system of the present disclosure obtain feature information and structure information of an input image, determine a prediction method and a quantization parameter based on the feature information and the structure information, and image-encode the input image based on the compression method and the quantization parameter, wherein the feature information includes at least one of a type of an image for the input image, a behavior of an object in an image or configuration information of an image, and the structure information may be information obtained by classifying areas of the input image into a video conference image, an X-ray image and a CT image based on the feature information.

### Mode for Invention

Hereinafter, referring to attached drawings, the embodiment of the present invention will be described in detail so that those skilled in the art may easily implement it in the technical field to which the present invention belongs. However, the present invention may be implemented in different forms and is not limited to embodiments described herein. In addition, in order to clearly explain the present invention in drawings, parts that are not related to the description are omitted, and similar drawing signs are attached to similar parts throughout

Throughout the specification, when a part is said to be connected to another part, it includes not only a case where it is directly connected, but also a case where it is electrically connected with other elements in between. In addition, when a part is said to include a component, it means that instead of excluding other components, other components may be further included, unless otherwise specifically opposed.

Throughout the specification, when a part is said to include a component, it means that instead of excluding other components, other components may be further included, unless otherwise specifically opposed. The term of degree such as ‘step for ~’ or ‘step of ~’ used throughout the specification does not mean a step for ~.

In addition, although terms ‘first’, ‘second’, etc. may be used to describe various components, the components should not be limited by the terms. The terms are used only to distinguish one component from other components.

In addition, as construction units shown in an embodiment of the present disclosure are independently shown to represent different characteristic functions, it does not mean that each construction unit is composed in a construction unit of separate hardware or one software. In other words, as each construction unit is described by being enumerated as each construction unit for convenience of a description, at least two construction units of each construction unit may be combined to form one construction unit or one construction unit may be divided into a plurality of construction units to perform a function. An integrated embodiment and a separate embodiment of each construction unit are also included in a scope of a right of the present disclosure unless they are beyond the essence of the present disclosure.

First, terms used herein are briefly described as follows.

A video decoding apparatus described below may be a device included in a server terminal such as a personal computer (PC), a laptop computer, a portable multimedia player (PMP), a wireless communication terminal, a smart phone, a TV application server, a service server, etc., and may refer to various devices including a user terminal such as various devices, a communication device such as a communication modem, etc. for communicating with a wired or wireless communication network, a memory for storing various programs and data for decoding an image or performing inter or intra prediction for decoding and a microprocessor for executing a program and calculating and controlling the program.

In addition, an image encoded into a bitstream by an encoder may be transmitted to an image decoding apparatus through a wired or wireless communication network such as the Internet, a wireless local area network, a wireless LAN network, a wibro network, a mobile communication network, etc. or through various communication interfaces such as a cable, a universal serial bus (USB), etc. in real time or non-real time and may be decoded, reconstructed and played back as an image.

Scalable Video refers to a video that hierarchically configures a compressed bitstream so that decoding may be performed at any bit rate. A single-layer decoding apparatus decodes only one bitstream that supports only one bit rate, frame rate and image size, while a decoding apparatus for a multi-layer video may support scalability for various bit rates, frame rates and image sizes.

The Scalable Video Coding (SVC) standard decodes one bitstream into multiple video layers, and each layer has its own bit rate, frame rate, image size and image quality. In other words, one bitstream may be composed of a base layer and scalable enhancement layers. Generally, an enhancement layer may be encoded to have higher image quality than a video created with previous base layers, and as a term used herein, a scalable video decoding apparatus may include a multi-layer video decoding apparatus.

The general meaning of Dynamic Range (DR) refers to a difference between the maximum signal and the minimum signal that may be measured simultaneously in a measurement system. In image processing and video compression fields, a dynamic range may refer to the range of brightness that may be expressed by an image.

Standard Dynamic Range (SDR) has a contrast ratio of 1,000:1 and the maximum brightness of 100 nits, which is generally called a standard contrast ratio.

High Dynamic Range (HDR) generally refers to a high contrast ratio of 100,000:1 or more and has the maximum brightness of 4,000 nits. In addition, it corresponds to a brightness range that human eyes may see without luminance adaptation.

Enhanced Dynamic Range (EDR) refers to a contrast ratio between SDR and HDR (1,000:1 or more to less than 100,000:1) and has the maximum brightness of 1,000 nits.

In addition, a HDR image used herein refers to an image having a high dynamic range, and may include an image having a dynamic range of HDR and EDR as a concept contrasting with a SDR image.

Typically, a video may be composed of a series of pictures, and each picture may be partitioned into a high-level coding structure such as a slice and a tile and a coding unit in the form of a block such as a CTB, a PB and a CB. In addition, according to an embodiment, a coding structure and a block may also be partitioned into a circle or an irregular form as well as a polygonal form such as a triangle, a rhombus, a parallelogram, etc. not a square or a rectangle.

Those skilled in the art will understand that a term ‘Picture’ described below may be used by being replaced with other terms having an equivalent meaning such as an image, a frame, etc.

Hereinafter, the embodiment of the present invention will be described in more detail by referring to attached drawings. In describing the present invention, the overlapping description of the same components is omitted.

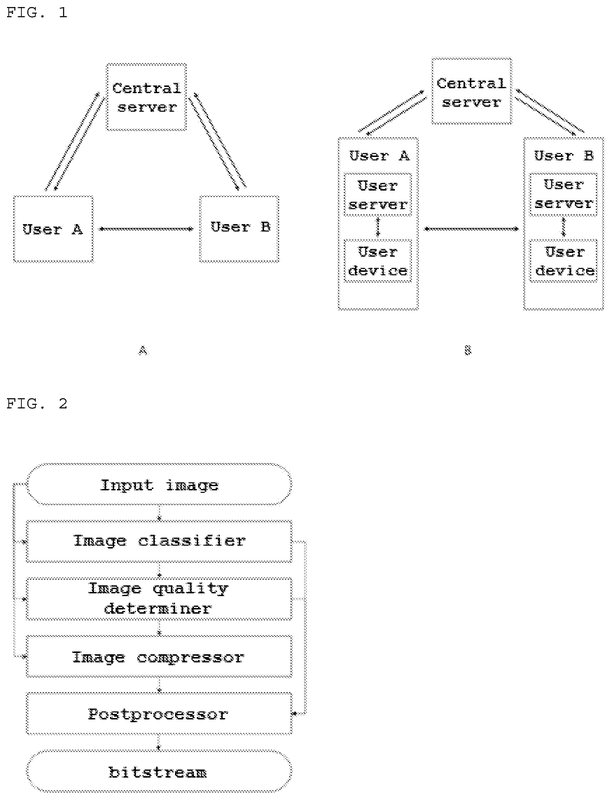

FIG. 1 conceptually shows a remote medical system according to an embodiment of the present invention. The remote medicine system may be typically used for medical communication between users such as between a medical professional and a medical professional or between a medical professional and a patient. The embodiment of the present invention assumes 1:1 to describe an embodiment with a simplified structure, but it may be performed in the form of N:M as well as 1:1 according to an embodiment. A proposed method may support not only direct communication between users, but also indirect communication through the central server of the remote medicine system and a separate communication structure where each user receives data for remote medicine. Alternatively, according to the characteristics of a user's device, a separate user server only for a user may be provided and a central server may communicate with a user server or communication between user servers may proceed and each user device may communicate with a user server. In this case, a user's server or a user device and a central server may learn and store or receive and store additional information for encoding/decoding and rendering in order to effectively encode/decode a video and use corresponding information to perform encoding/decoding and rendering.

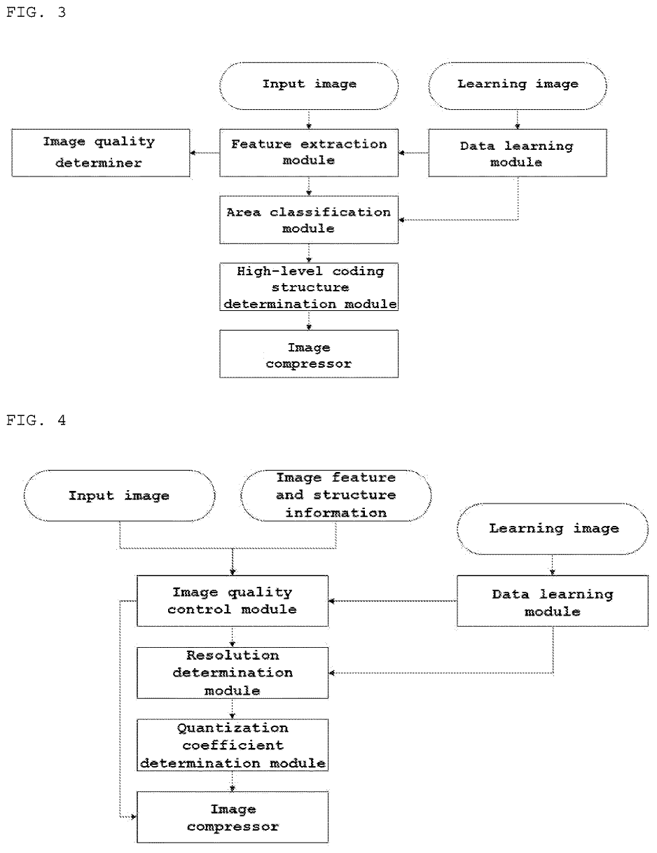

FIG. 2 is a block diagram of a system for image processing in a proposed system. An image processing system according to this embodiment may be operated in the user device or the user server of FIG. 1, and according to an embodiment, some of the processing steps of the system may be distributed and operated in at least one apparatus of a central server, a user device and a user server. In a proposed embodiment, when an image is input, an input image is input to an image classifier and classified based on the feature information of an image, and area partition information and a high-level coding structure are determined according to the feature information of an image. Extracted feature information is transmitted to an image quality determiner, and area partition information is transmitted to an image encoder. An image quality determiner determines information on the quantization coefficient and the image quality of an image to be input to an image encoder based on the feature information of an image received from a feature extractor. In this case, preprocessing filtering may be performed on an input image according to an embodiment. An image encoder performs actual video compression and generates a bitstream based on information on a high-level coding structure received from an image classifier and image quality and a quantization coefficient received from an image quality determiner.

FIG. 3 is a block diagram specifically showing an image classifier among each step of the system proposed in FIG. 2. The feature extraction module of an image classifier extracts the feature of an image based on learning data previously learned for an input image. In this case, learned data may be information stored in at least one apparatus of a user device, a user server and a central server. And, learned data may be updated fully or partially through the data of a newly input input image and then stored and transmitted again. In an embodiment, a feature extracted through a feature extraction module refers to the type of an image, the behavior of an object in an image, the configuration information of an image, etc. For example, it is determined and interpreted that a current image includes which data and consists of which image data among the image date that may be shared through remote medicine such as a general video conference image between users, a medical image such as MRI, CT and X-ray, a text information image about a patient's medical information, a medical image such as ultrasound/endoscopy, a surgery/procedure image, etc. According to an embodiment, an input image may be composed of at least one image among the medical image information, and a feature extraction module may extract information on whether it is a single data image or a multi-data image. When the configuration information of data is extracted through a feature extraction module, corresponding extraction information is input to an area partition module. An area partition module classifies the area of data having the same feature based on configuration information and pre-learned learning data. For example, when an input image is composed of a video conference image between users, a text image on user's medical information and an X-ray image, a structure in which three images are arranged and boundary information thereof are extracted. As another example, when an input image is composed of a video conference image between users, an X-ray image and a CT image, it may be classified into two areas, a video conference image and a medical image, or may be classified into three areas, a video conference image, an X-ray image and a CT image, according to an embodiment to extract structure information thereof. Through structure information to be extracted in this way, a high-level coding structure determination module determines a high-level coding structure according to the type of a video encoder/decoder currently used in a system. A high-level coding structure is a partition structure higher than a unit where actual encoding/decoding is performed, and may refer to a concept, for example, such as a picture, a sub-picture, a tile, a slice, etc., and may exist under a different name with a similar concept according to the compression technology of an encoder/decoder.

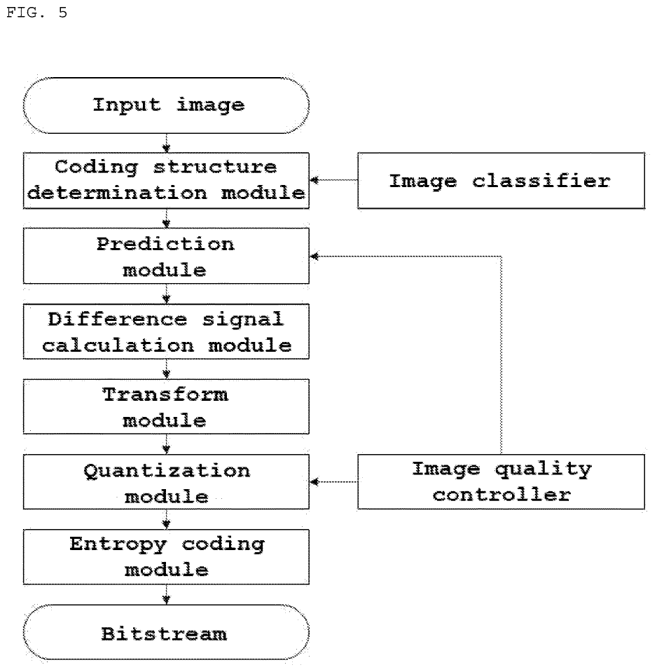

FIG. 4 is a block diagram specifically showing an image quality determiner among each step of the system proposed in FIG. 2. In the proposed invention, an image quality determiner determines the encoded image quality of an input image based on the feature information and structure information of an image determined and extracted by a feature extraction module and an area partition module in FIG. 3. In a general video compression system, the encoded image quality of an image may be adjusted through the ratio of the chroma and luma component of an image, a quantization coefficient, a resolution, etc., and since the deterioration of the encoded image quality may be refined through some filtering, it may also be adjusted through whether filtering exists or the number of filters and a coefficient. In a proposed method, an image quality determination module determines whether the lossy compression of an image exists, a compression ratio, a prediction method during image compression, etc. according to an embodiment based on input information. In this case, pre-learned learning data is used, and in this case, learned data may be information stored in at least one apparatus of a user device, a user server and a central server. When whether lossy compression exists and a compression ratio are determined in an image quality control module for an image area, a resolution determination module determines the resolution of each area. A prediction method is transmitted to an image compressor. A resolution determination module determines information on at least one of whether to change a resolution for whether each image area will perform compression at the same resolution as an input image or whether it will perform compression by increasing or decreasing a resolution and the type of a filter to be applied for changing a resolution when a resolution is changed. For a determination, whether to change a resolution and the type of a filter to be used for changing a resolution may be determined based on the learning information of a pre-learned image. The next quantization coefficient determination module determines the initial value of a quantization coefficient to be applied at a quantization step in an image compressor for each image area based on information determined in an image quality control module and a resolution determination module and transmits it to an image compressor. For example, when it is determined that lossless compression is performed at the same resolution in some areas of an image and lossy compression is performed at a resolution downsampled by ½ in horizontal and vertical directions, respectively, in the remaining areas, the type of a filter and the coefficient of a filter for downsampling a corresponding area are determined to perform downsampling. The initial coefficient of quantization is determined for each of an area where lossless compression is performed and an area where lossy compression is performed and a corresponding coefficient is transmitted to an image compressor.

FIG. 5 is a block diagram specifically showing an image compressor among each step of the system proposed in FIG. 2. An image compressor determines a high-level coding structure based on information received from an image classifier and partitions an image in a unit where actual encoding will be performed based on a determined high-level coding structure. A prediction module performs prediction encoding on image information partitioned in this way by using a prediction method received from an image quality controller. A prediction method received from an image quality controller is information including intra prediction, inter prediction, intra block copy, a mixed prediction method of intra and inter prediction, information on the number of reference images in inter prediction, etc. When prediction is performed in this way, a difference signal calculation module decodes a prediction signal and calculates a difference signal on a decoded signal and an original signal and a transform module performs transform on a difference signal. Afterwards, a quantization module performs quantization on a transformed difference signal coefficient by using a quantization parameter received from an image quality controller. According to an embodiment, a quantization parameter received from an image quality controller may be applied only to the initial block of each area and a subsequent block may be changed and applied by the rate control algorithm of an encoder or the same quantization parameter may be applied to a corresponding area. A coefficient quantized in this way is entropy encoded in an entropy coding module to generate a bitstream and is transmitted to a decoder. A general image decoder generates a decoded image signal by applying a bitstream received from an encoder in the reverse order of an encoding method. In a proposed method, when the same learning data is stored in the server or device of users or may be transmitted from a central server, a high-level coding structure transmitted to an image compressor through an image classifier and an image quality controller and the initial quantization parameter of each area may be omitted without being encoded.

The exemplary methods of the present disclosure are described as a series of operations for clarity of explanation, but this is not intended to limit the order in which the steps are performed, and when necessary, each step may be performed simultaneously or in a different order. In order to implement a method according to the present disclosure, another step may be additionally included in an exemplary step or the remaining steps may be included excluding some steps or another additional step may be included excluding some steps.

The various embodiments of the present disclosure do not list all possible combinations, but are intended to describe the representative aspect of the present disclosure, and matters described in various embodiments may be applied independently or in a combination of at least two.

In addition, the various embodiments of the present disclosure may be implemented by hardware, firmware, software or a combination thereof. For implementation by hardware, they may be implemented by one or more ASICs (Application Specific Integrated Circuits), DSPs (Digital Signal Processors), DSPDs (Digital Signal Processing Devices), PLDs (Programmable Logic Devices), FPGAs (Field Programmable Gate Arrays), general processors, controllers, microcontrollers, microprocessors, etc.

The range of the present disclosure includes software or machine-executable instructions (i.e., an operating system, an application, firmware, a program, etc.) that enable operations according to the methods of various embodiments to be executed on a device or computer, and a non-transitory computer-readable medium in which such software or instructions are stored and executable on a device or computer.

### INDUSTRIAL APPLICABILITY

The invention of the present disclosure may be utilized in video conference and medical fields.

1. A video compression and transmission device for a remote medical system, the device comprising:
an image classifier for obtaining feature information and structure information of an input image;
an image quality controller for determining a prediction method and a quantization parameter based on the feature information and the structure information; and
an image encoder for image-encoding the input image based on the compression method and the quantization parameter,
wherein the feature information includes at least one of a type of an image for the input image, a behavior of an object in an image or configuration information of the image, and
wherein the structure information is information obtained by classifying areas of the input image into a video conference image, an X-ray image and a CT image based on the feature information.

2. The device of claim 1, wherein:
an image classifier determines a coding structure of a higher level than a unit in which the image is encoded in the image encoder based on the feature information and the structure information.

3. The device of claim 2, wherein:
the higher level is any one of a picture, a sub-picture, a tile and a slice.

4. The device of claim 3, wherein:
the image encoding is performed based on the coding structure of the higher level.

5. The device of claim 1, wherein the image encoder includes:
a prediction module for obtaining a prediction signal by predicting the input image based on the prediction method;
a difference signal calculation module for obtaining a difference signal by subtracting the prediction signal and an original signal of the input image;
a transform module for obtaining a transform signal by transforming the difference signal;
a quantization module for obtaining a quantization signal by quantizing the transform signal based on the quantization parameter; and
an entropy coding module for generating a bitstream by encoding the quantization signal.

6. The device of claim 1, wherein:
the prediction method is any one of an intra prediction, an inter prediction, an intra block copy and a mixed prediction method of the intra prediction and the inter prediction.

7. The device of claim 1, wherein:
the prediction method is determined by considering at least one of whether a resolution is changed, whether a lossy compression is performed according to whether the resolution is changed or a compression ratio.

8. A video compression and transmission method for a remote medical system, the method comprising:
obtaining feature information and structure information of an input image;
determining a prediction method and a quantization parameter based on the feature information and the structure information; and
image-encoding the input image based on the compression method and the quantization parameter,
wherein the feature information includes at least one of a type of an image for the input image, a behavior of an object in an image or configuration information of the image, and
wherein the structure information is information obtained by classifying areas of the input image into a video conference image, an X-ray image and a CT image based on the feature information.

9. The method of claim 8, wherein:
the image encoding is performed based on a coding structure of a higher level than a unit in which the input image is encoded.

10. The method of claim 9, wherein:
the coding structure of the higher level is determined based on the feature information and the structure information.

11. The method of claim 10, wherein:
the higher level is any one of a picture, a sub-picture, a tile and a slice.

12. The method of claim 8, wherein:
the prediction method is any one of an intra prediction, an inter prediction, an intra block copy and a mixed prediction method of the intra prediction and the inter prediction.

13. The method of claim 8, wherein:
the prediction method is determined by considering at least one of whether a resolution is changed, whether a lossy compression is performed according to whether the resolution is changed or a compression ratio.

14. A computer-readable recording medium for storing a bitstream generated by a video compression and transmission method for a remote medical system, wherein the video compression and transmission method for the remote medical system includes:
obtaining feature information and structure information of an input image;
determining a prediction method and a quantization parameter based on the feature information and the structure information; and
image-encoding the input image based on the compression method and the quantization parameter,
wherein the feature information includes at least one of a type of an image for the input image, a behavior of an object in an image or configuration information of the image, and
wherein the structure information is information obtained by classifying areas of the input image into a video conference image, an X-ray image and a CT image based on the feature information.
